(19) United States
(12) Patent Application Publication
(10) Pub. No.: US 2022/0401006 A1
(43) Pub. Date: Dec. 22, 2022

(54) MEDICAL OR SURGICAL EQUIPMENT

(71) Applicant: Wolfram LAMADÉ, Bad Soden (DE)

(72) Inventor: Wolfram LAMADÉ, Bad Soden (DE)

(21) Appl. No.: 17/642,930

(22) PCT Filed: Sep. 14, 2020
(86) PCT No.: PCT/EP2020/075614

§ 371 Date: Mar. 14, 2022

(30) Foreign Application Priority Data
Sep. 13, 2019 (DE) 10 2019 124 719.5

(51) Int. Cl. A61B 5/388; A61B 5/00

(52) U.S. Cl. CPC A61B 5/388; A61B 5/6877; A61B 2560/0412

(57) ABSTRACT

In a medical or surgical equipment for receiving signals or for outputting signals from or to organic signal transmitters or receivers (4), such as in particular nerves, wherein on or in at least one carrier strip (1) at least one signal transmitter (2) for signals from or signals to the organic signal transmitter or receiver (4) is provided, which can be brought into contact with the organic signal transmitter or receiver (4), the carrier strip (1) is intended to change its shape when there is a change in a medium surrounding it or in a medium present in it or by a medium that can be introduced into it, in such a way that it adapts to the organic signal transmitter or receiver (4).

(FIG. 2)

The invention relates to medical or surgical equipment for receiving signals or for outputting signals from or to organic signal transmitters or receivers, such as in particular nerves, wherein on or in at least one carrier strip at least one signal carrier for signals from or signals to the organic signal transmitter or receiver is provided, which can be brought into contact with the organic signal transmitter or receiver, and corresponding methods therefor.

### Prior Art

In many cases during medical or surgical procedures on the human or animal body, it is desirable to monitor, stimulate or otherwise treat certain parts of the human or animal body. In particular, these are fibers that can transmit or receive signals. The present invention relates to all such parts of the human or animal body, but particularly to nerves. For years, so-called neuromonitoring has been performed here, for example, to determine whether nerve structures located in the surgical area are injured. On the other hand, it is also known that nerves can be stimulated by electrical impulses, the response of which is in turn determined and analyzed. The present invention can be used in a variety of ways.

The US 2007/0043411 A1, for example, describes an electrode for application to the vagus nerve. For this purpose, it has a certain curve from the outset, to which a flexible extension is connected. Due to the predetermined curve, the contact area with the nerve is very limited.

The DE 20 2010 006 586 U1 also describes a sleeve electrode with a safety anchorage, which has proved very successful in practice. However, the application of this electrode is complicated and requires the hand of the surgeon. Furthermore, the contact area between the electrode and the nerve is also limited here.

### Object

The object of the present invention is to provide an equipment of the above type which can be easily put on by the surgeon, which has a large contact area with the organic signal transmitter or signal receiver, and which in turn can also be easily removed.

### Solution

The solution of the object is that the carrier strip changes its shape when there is a change in a medium surrounding it or in a medium present in it, or by a medium that can be introduced into it, in such a way that it adapts to the organic signal transmitter or receiver.

In many other applications, this change in shape is also referred to as the memory effect. For example, shape memory polymers or shape memory alloys are known which can apparently “remember” their former external shape despite a strong deformation in the meantime. Such materials are thus changed in shape by an external influence; if this influence is removed, they can return to their old shape. If, for example, such materials are used as coatings for materials that have no memory, they also take this material with them into the new shape and allow it to return to its old shape.

In the present case, the main idea is that the carrier strip winds around the organic signal transmitter or receiver in the shape of a spiral or helix. This means that the surgeon's hand is no longer required to apply the carrier strip to the signal transmitter or receiver, thus avoiding any hygienic influences or risk of injury. The carrier strip wraps itself around the signal transmitter or receiver without any intervention by the surgeon. Furthermore, it is of course of significant advantage that the wrapping of the signal transmitter or receiver is done very tightly, so that a very good contact between the carrier strip and the signal transmitter or receiver is established. The transmission of a signal is thereby substantially improved.

Preferably, the carrier strip is sickle-shaped or L-shaped. This means that when it is applied to the signal transmitter or receiver, it does not wrap around itself but forms a helix with the largest possible contact area with the signal transmitter or receiver. Furthermore, it is also thought of to let a preformed helical electrode slide out of a straight protective sheath, so that it can take on its helical shape afterwards. This is known from the so-called pigtail-catheters.

Furthermore, the carrier strip consists of a very thin, flexible material, almost like a foil, which wraps around the signal transmitter or receiver. To pull it off, it is sufficient, for example, to pull on a conductor to the carrier strip, so that this carrier strip then unwinds from the signal transmitter or receiver.

In a first embodiment, it is thought that the change in shape is achieved by a hydraulic or pneumatic device. For example, a channel for a hydraulic or pneumatic medium could be integrated in the carrier strip, which is designed in such a way that the carrier strip undergoes a change in shape when the medium is introduced. In this case, the corresponding hydraulic or pneumatic medium is located in a reservoir which is also integrated in the carrier strip. When this reservoir is pressurized, the hydraulic or pneumatic medium is forced into the channel that runs through the carrier strip. This straightens a previously spirally wound carrier strip. When the pressure on the reservoir is released, the medium flows back into the reservoir and the carrier strip returns to its original spiral or helical shape. This can, of course, be further enhanced by the fact that the carrier strip is made of a material with a memory effect.

In another embodiment of the invention, it is contemplated that the carrier strip is made of a material that undergoes a change in shape when the temperature changes. This is, for example, a material that maintains a flat shape when cooled, whereas it curves when the temperature is increased. For the present case, this means that the carrier strip is kept refrigerated prior to surgery for example, and is then removed from the cold room for use during surgery and inserted into the patient's tissue near the signal transmitter or receiver to be enclosed. Due to the heat, the carrier strip now curves and winds itself as a spiral or helix around the signal transmitter or receiver.

In certain cases, it may be necessary to carry out the temperature change for the carrier strip in a controlled manner. In this case, it is provided according to the invention that a heat element is assigned to the carrier strip. For example, a resistance wire could be integrated in the carrier element. This makes the equipment according to the invention independent of external heat supply. Furthermore, the surgeon can determine exactly when he or she desires the enclosure of a signal transmitter or receiver. All possible heat elements are to be covered by the invention. Of course, the carrier strip could also be irradiated with heat from the outside, for example by an infrared lamp or the like. This could of course also heat the tissue around the carrier strip, so that the tissue can better transfer the heat to the carrier strip.

In another embodiment of the invention, it is contemplated that the carrier strip is made of or includes a material that undergoes a change in shape or results in a change in shape of the carrier strip when an electrical pulse is applied or transmitted thereto. For example, metallic arrangements can be provided in the carrier strip which, when an electrical potential is applied to the carrier strip, lead to its curving. It is also possible to use a shape memory alloy for the carrier strip itself, in which case, of course, the electrodes used to receive or transmit signals from or to the signal transmitter or receiver must be isolated therefrom. All this is said to be covered by the idea of the invention.

The invention also comprises a method for performing a medical or surgical operation in which signals from or to an organic signal transmitter or signal receiver are received or output by medical or surgical equipment, wherein at least one signal carrier for signals from or signals to the organic signal transmitter or signal receiver is provided on or in at least one carrier strip of this equipment and this is brought into contact with the organic signal transmitter or signal receiver. In this process, the shape of the carrier strip for application to the organic signal transmitter or receiver is to be changed. This can be done by assigning a hydraulic or pneumatic device to the carrier strip, which enables the shape of the carrier strip to be changed.

Furthermore, a temperature of the carrier strip can be changed before its use in the surgery during the use, which leads to a change in the shape of the carrier strip, in particular to the application of the carrier strip to the signal transmitter or receiver. It is particularly preferred that the carrier strip is cooled prior to its use in the surgery.

Also conceivable is a method for performing a medical or surgical operation in which signals from or to an organic signal transmitter or signal receiver are received or output by medical or surgical equipment, wherein at least one signal carrier for signals from or signals to the organic signal transmitter or signal receiver is provided on or in at least one carrier strip of this equipment and this is brought into contact with the organic signal transmitter or signal receiver. When the carrier strip is applied to the organic signal transmitter or receiver, an electrical pulse causes a change in shape, in particular to an application of the carrier strip to the organic signal transmitter or receiver.

### DETAILED DESCRIPTION OF THE FIGURES

Further advantages, features and details of the invention result from the following description of preferred embodiment and from the drawing; these show in

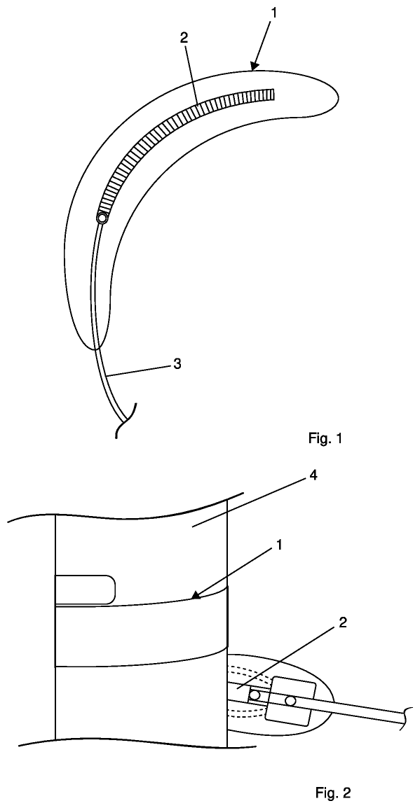

FIG. 1 a top view of a medical or surgical equipment according to the invention outside the position of use;

FIG. 2 a perspective view of the medical or surgical equipment according to the invention in the position of use;

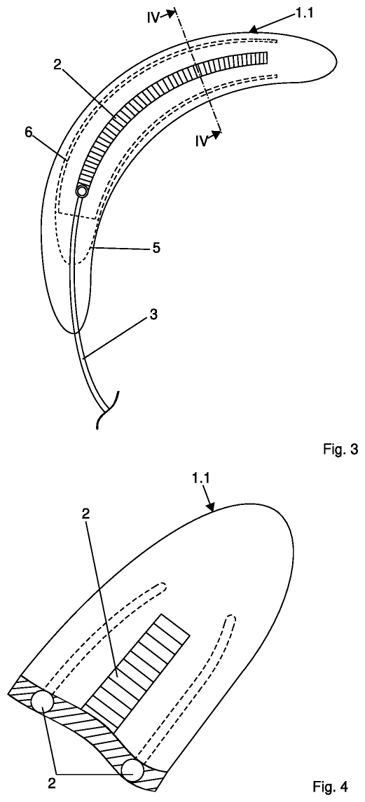

FIG. 3 a perspective view of a further embodiment of the medical or surgical equipment according to the invention outside the position of use;

FIG. 4 a perspective view of the medical or surgical equipment according to FIG. 3 along line IV-IV;

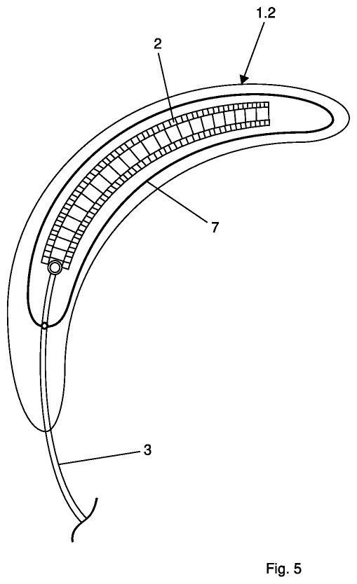

FIG. 5 a perspective view of a further embodiment of the medical or surgical equipment according to the invention outside the position of use.

According to FIG. 1, a medical or surgical equipment according to the invention has a carrier strip 1 which is covered with a signal transmitter 2. As a rule, the signal transmitter 2 is an electrode.

A conductor 3 to any electrical device is connected to the signal transmitter 2, with which a pulse of the signal transmitter can be received or a pulse can be output to the signal transmitter 2.

The functioning of the present invention is as follows:

An essential feature of the present invention is the design of the carrier strip 1, which, as shown in FIG. 2, should be able to change its shape in such a way that it can be wrapped around a nerve 4, for example, as an example of an organic signal transmitter or receiver. The preferred L-shape (or also sickle, banana or boomerang shape) of the carrier strip 1 also enables a helix-like wrapping around the signal transmitter or receiver 4, whereby the contact possibility is further increased.

In a first embodiment of the invention, the carrier strip 1 has the shape outlined in FIG. 1 and is kept refrigerated. However, it is made of a material that changes shape when the temperature changes. For example, the inner surface of the carrier strip 1 may be coated with a material that contracts when the temperature is increased, causing the carrier strip 1 to twist.

Thus, if the carrier strip 1 is applied to, for example, a nerve 4 during a surgery, a change in shape occurs as a result of the heating of the carrier strip 1 by the warmer tissue surrounding it and, in particular, a twisting of the carrier strip so that it wraps around the nerve 4.

However, the carrier strip 1 is made of such a flexible material that the change in shape can be overcome by pulling lightly on the conductor 3 and the carrier strip can be pulled off the nerve 4.

In a further preferred embodiment according to FIGS. 3 and 4, a hydraulic or pneumatic device is integrated in a carrier strip 1.1. This consists of a reservoir 5 and at least one, preferably two, conduits 6.1 and 6.2 for a hydraulic or pneumatic medium. This runs along the carrier strip 1.1 and the signal transmitter 2. When pressure is applied to the reservoir 5, a hydraulic or pneumatic medium flows into the conduit 6.1, 6.2 and causes the carrier strip 1.1 to align straight and flat. When the pressure on the reservoir 5 is released, the hydraulic or pneumatic medium flows back into the reservoir 5 and the carrier strip 1.1 returns to its original helically wound shape. This can be assisted, for example, by the carrier strip 1.1 being made of a material with a memory effect.

If the equipment according to the invention is thus required during a surgery, for example on a nerve 4, the surgeon can press on the reservoir 5, for example with tweezers, causing the hydraulic or pneumatic medium to flow into the conduit 6.1, 6.2 and straighten the carrier strip 1.1. Now the surgeon can place the carrier strip 1.1 against the nerve 4, release the pressure on the reservoir 5 and the carrier strip 1 wraps around the nerve 4.

When removing the equipment from the nerve 4, it is usually sufficient for the carrier strip 1.1 to be flexible enough to unroll from the nerve 4 when the conductor 3 is pulled. If this is not sufficient, the surgeon can again apply pressure to the reservoir 5 so that the carrier strip 1.1 straightens.

In a further embodiment of the invention according to FIG. 5, it is intended to integrate a resistance wire 7 in the carrier strip 1.2, via which heat can be supplied to the carrier strip 1.2 in a controlled manner. The control line required for this is also integrated in the conductor 3. This would allow the embodiment according to FIG. 1 to be better controlled, which is done by controlling the heat. However, the resistance wire 7 could also cause interference with the signal transmission. However, since very effective shielding of conductors is known today, this embodiment example is also to be covered by the invention.

Reference symbol list

| | | |
|---|---|---|
| 1 | Trägerstreifen | carrier strip |
| 2 | Elektroden | signal transmitter |
| 3 | Leiter | conductor |
| 4 | Nerv | nerve |
| 5 | Reservoir | reservoir |
| 6 | Leitung | conduit |
| 7 | Widerstandsdraht/ | resistance wire/ |
| | Wärmeelement | heating element |

1. Medical or surgical equipment for receiving signals or for outputting signals from or to organic signal transmitters or receivers (4), such as in particular nerves, wherein on or in at least one carrier strip (1, 1.1, 1.2) at least one signal carrier (2) for signals from or signals to the organic signal transmitter or receiver (4) is provided, which can be brought into contact with the organic signal transmitter or receiver (4),
characterized
in that the carrier strip (1, 1.1, 1.2) changes its shape when there is a change in a medium surrounding it or in a medium present in it or by a medium which can be introduced into it, in such a way that it adapts to the organic signal transmitter or receiver (4).

2. Equipment according to claim 1, characterized in that the carrier strip (1, 1.1, 1.2) winds around the organic signal transmitter or receiver (4) in the form of a spiral or helix.

3. Equipment according to claim 1, characterized in that the carrier strip (1.1) is assigned with a hydraulic or pneumatic device which effects the change in shape of the carrier strip (1.1).

4. Equipment according to claim 3, characterized in that in the carrier strip (1.1) a reservoir (5) for a hydraulic or pneumatic medium as well as at least one conduit (6.1, 6.2) connected to this reservoir (5) is integrated.

5. Equipment according to claim 4, characterized in that the conduit (6.1, 6.2) runs away from the reservoir (5) and at least along the signal transmitter (2).

6. Equipment according to claim 1, characterized in that the carrier strip (1,1.2) consists of a material which undergoes a change in shape when the temperature changes.

7. Equipment according to claim 1, characterized in that the carrier strip (1.2) consists of a material or contains a substance which undergoes a change in shape or leads to a change in shape of the carrier strip (1.2) when an electrical pulse is applied or transmitted.

8. Equipment according to claim 1, characterized in that the carrier strip (1, 1.1, 1.2) is L-shaped.

9. Equipment according to claim 1, characterized in that a heating element (7) is integrated in the carrier strip (1, 1.1, 1.2).

10. Method for performing a medical or surgical operation, in which signals from or to an organic signal transmitter or signal receiver (4) are received or output by medical or surgical equipment, wherein at least one signal carrier (2) for signals from or signals to the organic signal transmitter or signal receiver (4) is provided on or in at least one carrier strip (1, 1.1, 1.2) of this equipment and this is brought into contact with the organic signal transmitter or signal receiver (4), characterized in that the shape of the carrier strip (1, 1.1, 1.2) for application to the organic signal transmitter or signal receiver (4) is adapted to the shape of the organic signal transmitter or signal receiver (4), characterized in that the shape of the carrier strip (1, 1.1, 1.2) is changed for application to the organic signal transmitter or receiver (4).

11. Method according to claim 10, characterized in that the shape of the carrier strip (1.1) is changed by a hydraulic or pneumatic device associated with the carrier strip (1.1).

12. Method according to claim 10, characterized in that a temperature of the carrier strip (1) is changed before its use in the surgery during the use, which leads to a change in shape of the carrier strip (1), in particular to the application of the carrier strip (1) to the signal transmitter or receiver (4).

13. Method according to claim 12, characterized in that the carrier strip (1) is cooled prior to its use in the surgery.

14. Method for performing a medical or surgical operation, in which signals from or to an organic signal transmitter or signal receiver (4) are received or output by medical or surgical equipment, wherein at least one signal transmitter for signals (2) from or signals to the organic signal transmitter or signal receiver (4) is provided on or in at least one carrier strip (1.2) of this equipment and this is brought into contact with the organic signal transmitter or signal receiver (4), characterized in that, when the carrier strip (1.2) is applied to the organic signal transmitter or receiver (4), an electrical pulse causes a change in shape, in particular to an application of the carrier strip (1.2) to the organic signal transmitter or receiver (4).

15. Equipment according to claim 2, characterized in that the carrier strip (1.1) is assigned with a hydraulic or pneumatic device which effects the change in shape of the carrier strip (1.1).
